(19) United States
(12) Patent Application Publication
(10) Pub. No.: US 2025/0099663 A1
(43) Pub. Date: Mar. 27, 2025

(54) SYSTEM FOR ON-DEMAND MANUFACTURING OF PARENTERAL SOLUTIONS

(71) Applicant: Regents of the University of Minnesota, Minneapolis, MN (US)

(72) Inventors: Natasha C. Wright, Minneapolis, MN (US); Ibrahim Yekinni, Minneapolis, MN (US); Benjamin Szot, Minneapolis, MN (US); Carolyn F. Bernemann, Minneapolis, MN (US)

(21) Appl. No.: 18/852,914

(22) PCT Filed: Apr. 4, 2023
(86) PCT No.: PCT/US2023/017403

§ 371 Date: Sep. 30, 2024

Related U.S. Application Data
(60) Provisional application No. 63/327,059, filed on Apr. 4, 2022.

(51) Int. Cl. A61M 1/28; A61J 1/10; A61J 1/14; A61J 1/20; A61J 1/22; A61L 2/10; A61L 2/18; A61L 101/02; A61L 101/34; A61L 101/36

(52) U.S. Cl. CPC A61M 1/287; A61J 1/10; A61J 1/1406; A61J 1/201; A61J 1/2086; A61J 1/2089; A61J 1/22; A61L 2/10; A61L 2/18; A61L 2/186; A61J 2200/70; A61L 2101/02; A61L 2101/34; A61L 2101/36; A61L 2202/11; A61L 2202/122; A61L 2202/24

(57) ABSTRACT

A system for manufacturing parenteral solutions includes a drug dosage module and a mixing module. The drug dosage module includes a source of one or more parenteral drug solutes, and one or more metering devices configured to discharge a metered dosage of each of the parenteral drug solutes. The mixing module includes a container configured to receive water-for-injection (WFI) and the parenteral drug solutes discharged from the one or more metering devices, and a mixer configured to mix water-for-injection (WFI) and the parenteral drug solutes in the container to form a parenteral solution.

### FIELD

Embodiments of the present disclosure relate to systems for manufacturing parenteral solutions, such as solutions for peritoneal dialysis for the treatment of renal insufficiency.

### BACKGROUND

Patients suffering from acute or chronic renal insufficiency are treated using dialysis therapy. One such dialysis therapy is peritoneal dialysis, in which a dialysis solution is infused into the patient's peritoneal cavity. Metabolites diffuse across the peritoneal membrane into the dialysis solution, which is then removed from the patient to complete the therapy.

Limited access to dialysis may result in up to 7 million deaths annually, more than the number of deaths from HIV and tuberculosis combined. Less than one-third of patients needing dialysis in Asia and only 16% of the patients in Africa receive needed treatment. This is due in part to the lack of local production of dialysis fluid in the countries affected; importation leads to high costs that make the solution unaffordable to patients who usually have to pay out of pocket.

Dialysis fluid is part of a larger category of treatment solutions referred to as Large Volume Parenteral Solutions (LVPS); others include infusion solutions (plasma expanders, rehydration fluids, nutritional and electrolyte solutions) and irrigation solutions. These solutions, which are vital for routine medical care, are all generics formulated from similar ingredients. Water-for-Injection (WFI) forms 90% or more of the volume of most LVPS. Solutes (salts and sugars) make up the rest of the volume. The similarity in formulation is the reason some physicians improvise dialysis fluid by mixing other LVPS together in situations where shortages are extreme.

Besides the WFI required for producing LVPS, conventional systems require a controlled environment (e.g., clean room), in which the parenteral solutes (e.g., salts and sugars) may be mixed with the WFI and stored in containers.

New LVPS manufacturing techniques are needed to meet growing demands for LVPS globally and locally. For example, the global demand for LVPS is growing with an increasing number of surgeries and hospitalizations for chronic conditions—the global market is predicted to grow at a CAGR of 7.08% to $11.939 billion by the end of 2025. In contrast to the demand, the supply for LVPS even in developed markets like the US is becoming limited as pharmaceutical companies either offshore their production or discontinue manufacturing in pursuit of higher-margin drugs. This leads to shortages of these vital solutions with the slightest supply chain disturbances (e.g., 2018 saline shortage with Hurricane Maria, 2020 dialysis fluid shortage in the US due to increased demands from COVID-19 patients with kidney damage).

Additionally, there is a need to provide kidney care in developing countries and in disaster situations or military fields where supply chains for essential LVPS might be disrupted or non-existent.

### SUMMARY

Embodiments of the present disclosure relate to systems for manufacturing parenteral solutions, various components of the system, and methods performed by the systems. One embodiment of the system includes a drug dosage module and a mixing module. The drug dosage module includes a source of one or more parenteral drug solutes, and one or more metering devices configured to discharge a metered dosage of each of the parenteral drug solutes. The mixing module includes a container configured to receive water-for-injection (WFI) and the parenteral drug solutes discharged from the one or more metering devices, and a mixer configured to mix water-for-injection (WFI) and the parenteral drug solutes in the container to form a parenteral solution.

In one example, the mixing module includes at least one conductivity sensor, each configured to sense a conductivity of the parenteral solution in the container and produce a conductivity output that is indicative of the sensed conductivity. In one embodiment, the system may include a controller that is configured to control the discharge of the metered dosage of each of the parenteral drug solutes to the container by the one or more metering devices based on each conductivity output produced by the at least one conductivity sensor.

In one embodiment, the system includes a water module having a water pump that is configured to deliver a flow of the WFI to the container. The controller is configured to control the delivery of the flow of the WFI to the container by the water pump based on each conductivity output produced by the at least one conductivity sensor.

In yet another embodiment, the at least one conductivity sensor includes a plurality of the conductivity sensors. Each of the conductivity sensors is positioned at a different height within the container. The controller controls the discharge of the metered dosage of each of the parenteral drug solutes by the one or more metering devices and the flow of the WFI to the container by the water pump based on the conductivity outputs.

In one embodiment, the water module of the system includes a water purifier configured to filter or sterilize a flow of water to produce the flow of the WFI. The water purifier may include a water filter, through which the flow of water travels, and/or an ultraviolet (UV) sterilization device comprising one or more UV light sources configured to expose the flow of water to UV light.

Another example of the system includes a bagging module that is configured to deliver a flow of the parenteral solution into each of one or more fluid storage bags through a corresponding fluidic coupling. In embodiment, each of the fluidic couplings includes a first coupler configured to receive the flow of the parenteral solution and a second coupler attached to the fluid storage bag. The first and second couplers are configured to cooperate to form the fluidic coupling. In one embodiment, the first coupler includes a needle, and the second coupler includes a septum, the first and second couplers include cooperating tubing fittings, or the first and second couplers include cooperating Luer fittings.

The bagging module may include a solution pump configured to drive the flow of the parenteral solution from the container into each of the one or more fluid storage bags through the corresponding fluidic coupling.

In one embodiment, the bagging module includes a fluidic coupling drive mechanism that is configured to move each of the first couplers relative to the second couplers between a retracted position, in which each of the first couplers is recessed from the corresponding second coupler, and a filling position, in which each of the first couplers cooperates with the corresponding second coupler to form the fluidic couplings.

In another embodiment, the bagging module further comprises a sterilizer configured to sterilize the first coupler and/or the second coupler. In one example, the sterilizer may include at least one sterilization chamber, wherein the first coupler and/or the second coupler of each fluidic coupling is contained in one of the sterilization chambers. One or more UV lamps are contained in each of the sterilization chambers and are configured to expose the first coupler and/or the second coupler of each fluidic coupling to UV light.

In another example, the sterilizer includes at least one sterilization chamber, each containing a sterilization fluid. The first coupler and/or the second coupler of each fluidic coupling is contained in one of the sterilization chambers and the sterilization fluid.

In one embodiment, the bagging module includes a plurality of the fluid storage bags and a plurality of the fluidic couplings. The first coupler of each fluidic coupling includes a needle, and the second coupler of each fluidic coupling includes a bag septum that is fluidically coupled to an interior cavity of the corresponding fluid storage bag. The fluidic coupling drive mechanism is configured to move the plurality of needles relative to the corresponding bag septa between a retracted position, in which each of the needles is recessed from the corresponding bag septum, and a filling position, in which each of the needles pierces the corresponding bag septum and is configured to deliver the flow of the parenteral solution to the interior cavity of the corresponding fluid storage bag.

In one embodiment, the parenteral solutes include solid drug solutes and/or liquid drug solutes.

One embodiment of the present disclosure is directed to a bagging module that is configured to fill one or more fluid storage bags with parenteral solution. In one example, the bagging module includes a source of parenteral solution, one or more fluid storage bags, each having an interior cavity, a fluidic coupling corresponding to each fluid storage bag to facilitate delivery of a flow of the parenteral solution from the source into the one or more fluid storage bags. Each fluidic coupling may include a first coupler configured to receive the flow of the parenteral solution and a second coupler attached to the fluid storage bag. The first and second couplers are configured to cooperate to form the fluidic coupling. In one example, the first coupler includes a needle, and the second coupler includes a septum. In another example, the first and second couplers include cooperating tubing fittings. In another example, the first and second couplers include cooperating Luer fittings.

The bagging module may include a solution pump that is configured to drive the flow of the parenteral solution from the source into each of the one or more fluid storage bags through the corresponding fluidic coupling.

In one embodiment, the bagging module includes a fluidic coupling drive mechanism configured to move each of the first couplers relative to the second couplers between a retracted position, in which each of the first couplers is recessed from the corresponding second coupler, and a filling position, in which each of the first couplers cooperates with the corresponding second coupler to form the fluidic couplings.

In one embodiment, the bagging module includes a sterilizer that is configured to sterilize the first coupler and/or the second coupler. In one example, the sterilizer includes at least one sterilization chamber, wherein the first coupler and/or the second coupler of each fluidic coupling is contained in one of the sterilization chambers. One or more UV lamps are contained in each of the sterilization chambers and are configured to expose the first coupler and/or the second coupler of each fluidic coupling to UV light.

In another example, the sterilizer includes at least one sterilization chamber, each containing a sterilization fluid. The first coupler and/or the second coupler of each fluidic coupling is contained in one of the sterilization chambers and the sterilization fluid.

Additional embodiments include methods for manufacturing the parenteral solution using embodiments of the system and a controller configured to control components of the system, and methods for delivering parenteral solution into one or more bags using a bagging module.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the Background.

### BRIEF DESCRIPTION OF THE DRAWINGS

FIG. 1 is a simplified diagram of a system that is configured to manufacture large volume parenteral solutions, in accordance with one or more embodiments of the present disclosure.

FIG. 2 is a simplified diagram of components of the water module, the drug dosage module and the mixing module, in accordance with one or more embodiments of the present disclosure.

FIG. 3 is a simplified diagram of an example of a final product produced by the system, in accordance with one or more embodiments of the present disclosure.

FIG. 4 is a simplified diagram illustrating the use of the final product of FIG. 3 in a peritoneal dialysis procedure, in accordance with embodiments of the present disclosure.

FIGS. 5 and 6 are simplified diagrams of examples of a bagging module, in accordance with embodiments of the present disclosure.

FIGS. 7A and 7B are simplified side views of an example of a bagging module, in accordance with embodiments of the present disclosure.

FIGS. 8A and 8B are simplified side views of an example of a bagging module respectively in retracted and filling positions, in accordance with embodiments of the present disclosure.

FIGS. 9A and 9B are simplified side and top views of an example of a bagging module, in accordance with embodiments of the present disclosure.

FIGS. 10 and 11 respectively are simplified side and isometric views of an example of the bagging module, in accordance with embodiments of the present disclosure.

### DETAILED DESCRIPTION OF ILLUSTRATIVE EMBODIMENTS

Embodiments of the present disclosure are described more fully hereinafter with reference to the accompanying drawings. Elements that are identified using the same or similar reference characters refer to the same or similar elements. The various embodiments of the present disclosure may, however, be embodied in many different forms and should not be construed as limited to the embodiments set forth herein. Rather, these embodiments are provided so that this disclosure will be thorough and complete, and will fully convey the scope of the present disclosure to those skilled in the art.

Some embodiments of the present disclosure facilitate small-scale, decentralized production of dialysis fluid or other LVPS in a manner that can significantly improve accessibility and affordability of dialysis fluid and other LVPS. In some embodiments, the water-for-injection is produced at or near the point of care and is mixed with the required concentrates to form the LVPS without the need for a cleanroom facility. As a result, embodiments of the present disclosure may provide significant improvements to the accessibility and affordability of dialysis fluid and other LVPS by facilitating timely, local and affordable manufacture of LVPS.

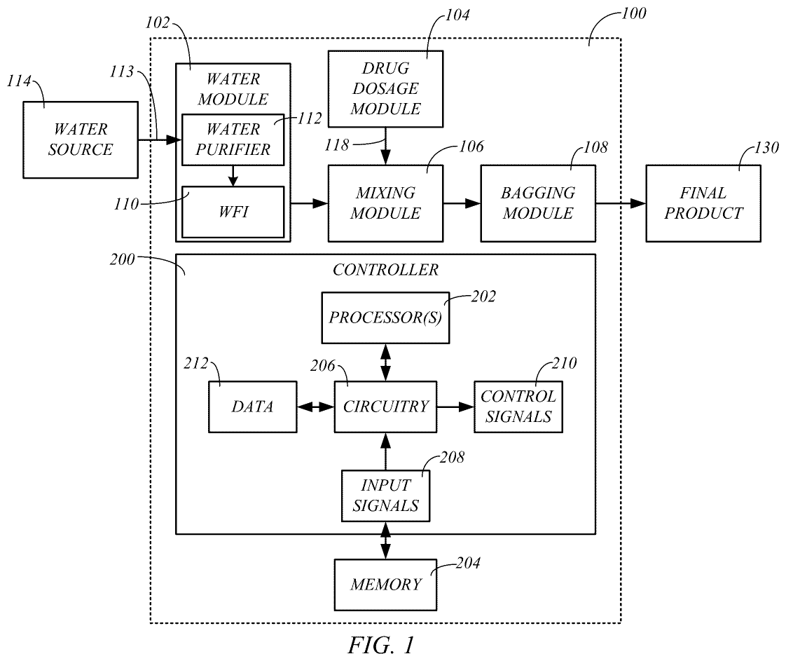

FIG. 1 is a simplified diagram of a system 100 that is configured to manufacture parenteral solutions, such as large volume parenteral solutions (LVPS), in accordance with one or more embodiments of the present disclosure. The parenteral solutions produced by the system 100 may include dialysis fluid, plasma expanders, rehydration fluids, nutritional and electrolyte solutions, irrigation solutions, and/or other parenteral solutions. The system 100 generally includes one or more modules that perform functions of the manufacturing process. In some embodiments, the modules of the system 100 include a drug dosage module 104 and a mixing module 106. Embodiments of the system 100 may also include a water module 102 and/or a bagging module 108.

The water module 102 is generally configured to supply purified water or water-for-injection (WFI) 110 to the mixing module 106. In one embodiment, the WFI 110 meets quality standards for injection, such as those defined by USP<1231> and/or other standards.

In some embodiments, the water module 102 may include a water purifier 112 that is configured to receive a supply of water from a water source 114. The water source 114 may be any suitable water source, such as line water from a community's supply of water, well water, or another suitable non-purified water source. The water purifier 112 purifies the water 113 received from the water source 114 using any suitable technique to form the WFI 110. In one example, the water purifier 112 includes one or more water filters, through which the water 113 travels, that remove impurities and sterilize the water 113 to form the WFI 110. Alternatively or additionally, the water purifier may include an ultraviolet (UV) sterilization or sanitization device having one or more UV light sources that expose the water 113 to UV light to sterilize the water 113 and form the WFI 110. The water purifier 112 may include other devices for sterilizing and sanitizing the water 113 from the water source 114 to form the WFI 110.

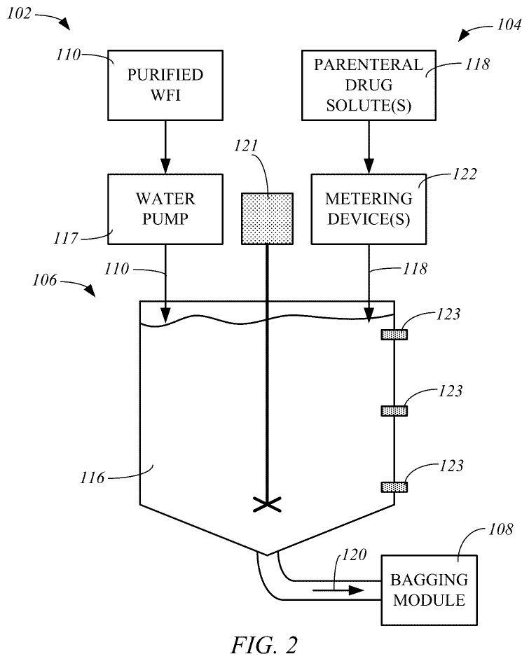

FIG. 2 is a simplified diagram of examples of the water module 102, the drug dosage module 104 and the mixing module 106, in accordance with one or more embodiments of the present disclosure. As indicated in FIG. 2, the mixing module 106 includes a tank or container 116. The volume of the container 116 may be selected to produce a desired volume of parenteral solution. In one example, the container 116 has a volume of 100 liters.

The water module 102 is configured to supply the WFI 110 to the container 116, as indicated in FIG. 2. In one example, metered amounts of the WFI 110 may be supplied to the container 116 using a conventional dosing pump 117 or another suitable technique.

The drug dosage module 104 is configured to supply the necessary sterile parenteral drug solutes or drug substances 118 to the container 116 of the mixing module 106 for mixing with the WFI 110 to form the desired parenteral solution 120, as indicated in FIG. 2. The drug solutes 118 may be in a concentrated solid and/or liquid form. Example parenteral drug solutes 118 include salts and sugars that are conventionally used to form parenteral solutions. The drug dosage module 104 may include one or more conventional metering devices 122 for delivering a desired dosage of each of the drug solutes 118 to the container 116.

The mixing module 106 may include a mixer 121 (FIG. 2) that is configured to mix or mechanically agitate the WFI 110 with the drug solutes 118 within the container 116 and/or recirculate the mixture to form the parenteral solution 120. The mixer 121 may take on any suitable form, such as a motorized mixing blade (shown), a magnetic stirring mechanism, or another suitable mixing device.

Some embodiments of the mixing module 106 are configured to monitor the parenteral solution 120 within the container 116 using one or more sensing elements 123. In one embodiment, the sensing elements 123 are configured to sense one or more parameters (e.g., conductivity, glucose, etc.) of the solution within the container 116, from which a concentration of the drug solutes 118 may be determined. In one embodiment, two or more of the sensors 123 are used to confirm that the solution is “fully mixed” and meets product specifications. This confirmation may be made without the use of external composition analyses. In one example, the sensors 123 are located at different heights along the interior of the container 116, as indicated in FIG. 2. This allows for parameter measurements at displaced locations of the solution 120. An average value of the sensed parameters may be used to assess whether sensed parameters of the solution 120 meet product specifications.

Some embodiments are directed to a process of forming the parenteral solution 120 using the water module 102, the drug dosage module 104 and the mixing module 106. In one example, the one or more sensors 123 comprise conductivity sensors that are configured to sense a conductivity of the solution 120 within the container 116. When the conductivity of the solution 120 within the container 116 detected by the one or more sensors 123 indicates that the concentration of the drug solutes 118 is less than the desired concentration, the drug dosage module 104 injects an incremental amount of one or more of the drug solutes 118 using the metering devices 122 and the solution 120 is retested using the sensors 123. When the concentration of the drug solutes 118 in the container 116 detected by the sensors 123 is greater than the desired concentration for the desired parenteral solution 120, the system 100 may inject an incremental amount of the WFI 110 using the dosage pump 117 of the water module 102. In this manner, the system 100 may produce a parenteral solution 120 within the container 116 having the desired concentration of the drug solutes 118, which may be output to the bagging module 108 using gravity or a pump, for example.

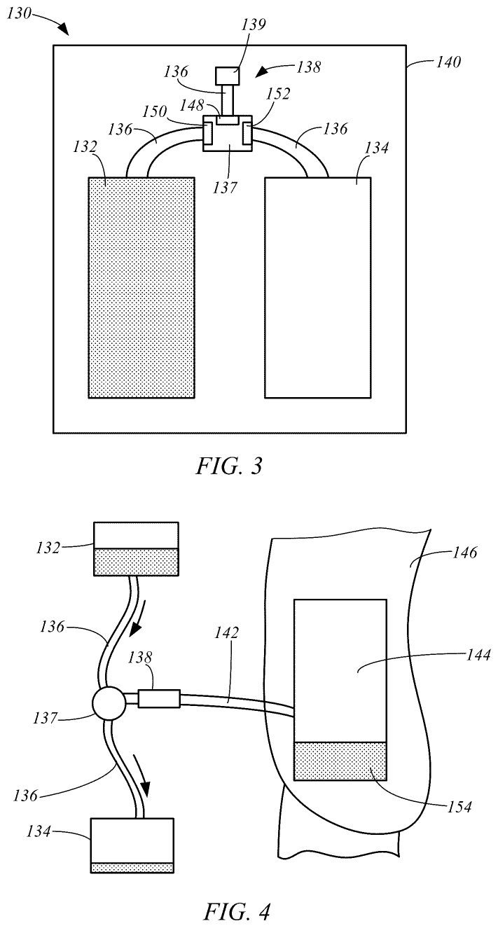

The bagging module 108 is generally configured to deliver a flow of the parenteral solution 120, such as from the mixing module 106, into one or more fluid storage bags, to produce a final product 130 (FIG. 1), which may be disposable. FIG. 3 is a simplified diagram of an example of the final product 130 that includes a primary container or bag 132 containing the parenteral solution 120. In some embodiments, the final product 130 also includes a drain bag 134, conduit sections 136, a valve or valving 137, and/or an adaptor 138. The adapter 138 may include a septum 139 and/or other conventional components. Packaging 140, such as a secondary bag, may contain the components of the final product 130. The bags 132 and 134 may represent malleable bags, rigid or semi-rigid containers, or other vestibules for liquid.

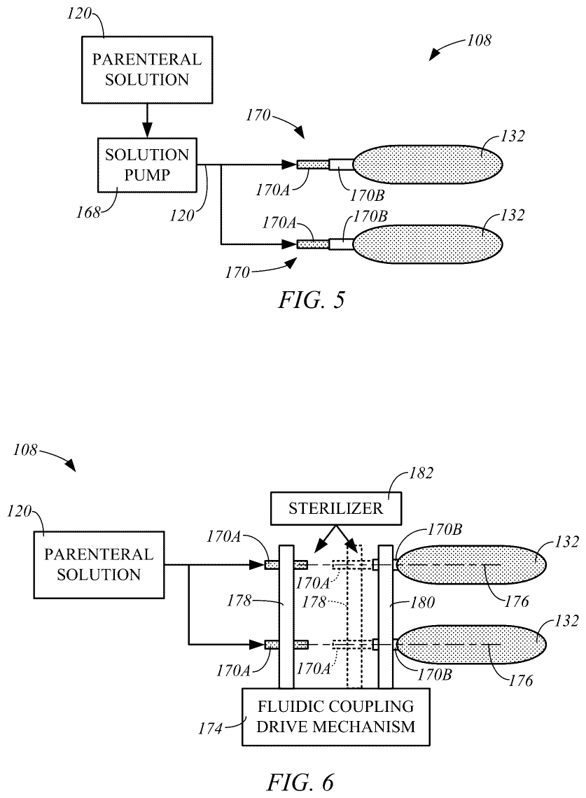

FIG. 4 is a simplified diagram illustrating the use of the final product 130 in a peritoneal dialysis procedure, in accordance with embodiments of the present disclosure. After removing the bags 132 and 134 and other components of the final product 130 from the package 140 (if present), the adaptor 138 may be connected to a catheter 142, which is fluidically coupled to the peritoneal cavity 144 of a patient 146. The adapter 138 may be connected to a port 148 of the valve 137 using a suitable conduit section 136. Additionally, a conduit section 136 may couple an output port of the primary bag 132 containing the parenteral solution 120 to a port 150 of the valve 137, and a conduit section 136 may couple an input port of the drain bag 134 to a port 152 of the valve 137.

The valve 137 may be set to a state in which a fluid pathway is formed between the port 150 connected to the primary bag 132 through the conduit 136 and the port 148 connected to the adapter 138 and catheter 142, while the port 152 connected to the drain bag 134 is closed to the fluid pathway. This allows the parenteral solution 120 in the form of dialysis fluid to flow from the bag 132 into the peritoneal cavity 144. After a suitable period of time has elapsed, the valve 137 may be set to a state that opens a fluid pathway between the port 148 connected to the adapter 138 and the catheter 142 and the port 152 connected to drain bag 134 through the conduit 136, while the port 150 connected to the primary bag 132 is closed to the fluid pathway. The fluid 154 within the peritoneal cavity 144 may then be drained through the catheter 142 and the tubing 136 to the drain bag 134 to complete the treatment. The product 130 may then be disposed of.

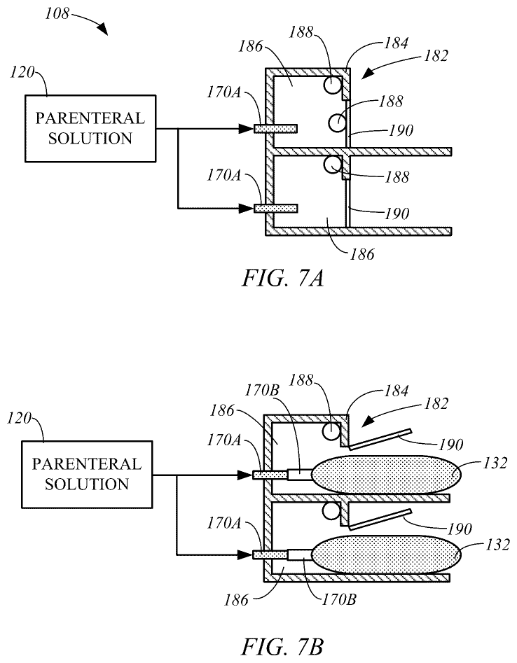

As mentioned above, the bagging module 108 is generally configured to deliver a flow of the parenteral solution 120 into one or more fluid storage bags 132. FIG. 5 is a simplified diagram of an example of the bagging module 108, in accordance with embodiments of the present disclosure.

In one embodiment, the bagging module 108 includes a solution pump 168 that may be used to drive a flow of the parenteral solution 120 (e.g., dialysis fluid), such as from the container 116 of the mixing module 106, into one or more fluid storage bags 132. Alternatively, the bagging module 108 may rely on gravity to deliver the flow of the parenteral solution 120 into the bags 132. While the bags 132 are generally shown as being in a horizontal orientation, embodiments of the bagging module 108 include orienting the bags 132 vertically to facilitate filing the bags 132 from above. Additionally, when multiple bags 132 are to be filled with the parenteral solution 120, the bagging module 108 may be configured to fill the bags 132 either in a serial or in a parallel manner.

In one embodiment, the bagging module 108 includes a fluidic coupling 170 that facilitates the formation of a fluid pathway for delivering the flow of the solution 120 into the bags 132. The fluidic coupling 170 generally comprises a first coupler 170A that receives the flow of the solution 120 and a second coupler 170B that is connected to a fluid pathway to the interior of the bag 132. The first and second couplers 170A and 170B cooperate with each other (e.g., connect, engage, etc.) to form the fluidic coupling 170. While only two fluidic couplings 170 and corresponding bags 132 are shown, it is understood that the bagging module 108 may be configured with one or several of the fluidic couplings 170 to meet bag filling requirements.

The fluidic couplings 170 may be formed using any suitable conventional couplings. In one example, the first coupler 170A includes a needle and the second coupler 170B comprises a suitable septum or sealable port. The needle form of the coupler 170A may be inserted into the septum or port form of the coupler 170B to open a fluid pathway for the delivery of the flow of the solution 120 into the bag 132.

In another example, the first and second couplers 170A and 170B may comprise cooperating tubing fittings. The tubing fittings may be conventional tubing fittings, such as suction connectors, rigid tubing sections, etc. For example, the first and second couplers 170A and 170B may comprise rigid tubing sections that allow one of the tubing sections to be received within the other to form the fluidic coupling 170 and the desired fluid pathway. Thus, the tubing section forming the first coupler 170A may have a diameter that is less than the tubing section forming the second coupler 170B to allow the first coupler 170A to be inserted into the second coupler 170B and form the fluidic coupling 170. Here, the tubing section forming the second coupler 170B and the bag 132 may be oriented vertically to allow the solution 120 to be pumped or gravity-fed into the bag 132 through the fluidic coupling 170.

In yet another example, the first and second couplers 170A and 170B may comprise conventional cooperating Luer components or fittings. For example, the first coupler 170A or the second coupler 170B may include a Luer fitting while the other includes a Luer activated valve. The fluidic coupling 170 that is completed when the Luer fitting is received in the valve forms the fluid pathway for the delivery of the solution 120 into the bag 132.

In some embodiments, the bagging module 108 may include a fluidic coupling drive mechanism 174, as illustrated in the simplified diagram of FIG. 6. The mechanism 174 generally operates to move the first couplers 170A relative to the second couplers 170B along their corresponding axes 176 between a retracted position (solid lines), in which the first and second couplers 170A and 170B are displaced from each other, and a filling position (phantom lines), in which the first and second couplers 170A and 170B form the fluidic coupling 170, such as that shown in FIG. 5. In some embodiments, the first couplers 170A may be connected to a structure 178 that is moveable by the mechanism 174 relative to a structure 180 supporting the second couplers 170B in alignment along the corresponding axis 176 with one of the first couplers 170A, as indicated in FIG. 6.

The drive mechanism 174 may take on any suitable form. In one example, the drive mechanism 174 may include a motor that drives a screw drive actuator, a rack and pinion actuator, or another suitable actuating mechanism for driving the structure 178 along the axis 176 relative to the structure 180. Alternatively, the drive mechanism 174 may be manually driven by a user.

In some embodiments, the bagging module 108 includes a sterilizer 182, as shown in FIG. 6. The sterilizer 182 operates to sterilize the first coupler 170A and/or the second coupler 170B, such as when the first couplers 170A are in the retracted position. The sterilizer 182 may take on various forms. For example, as discussed below, the sterilizer 182 may comprise one or more ultraviolet (UV) lamps that expose the first couplers 170A and/or the second couplers 170B to UV light at a sufficient dosage for sterilization, and/or one or more sterilization chambers containing a sterilization fluid to which the first couplers 170A and/or second couplers 170B are exposed. The sterilization fluid may include a chemical disinfectant, such as peracetic acid, hydrogen peroxide, isopropyl alcohol or another liquid disinfectant or sterilant.

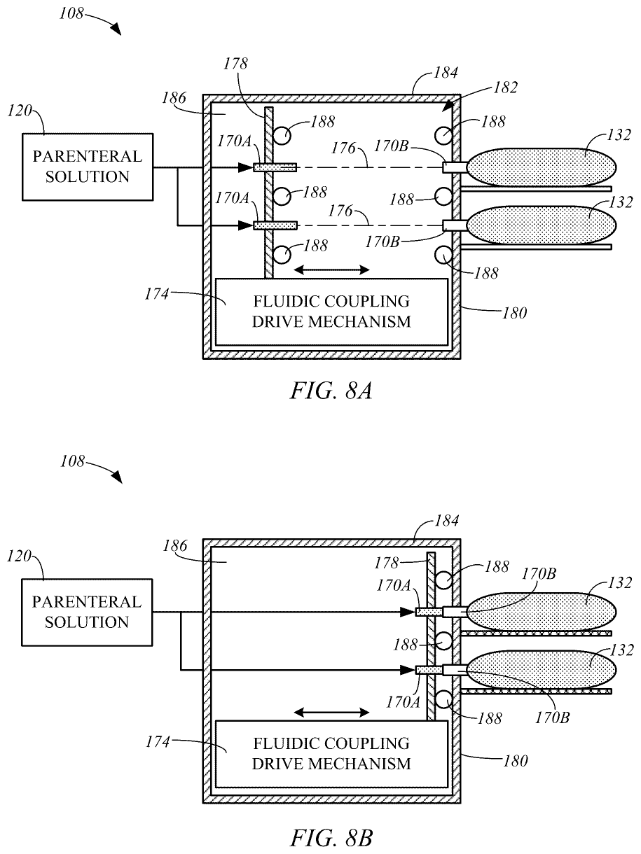

FIGS. 7A and 7B are simplified side views of an example of a bagging module 108, in accordance with embodiments of the present disclosure. In some embodiments, the bagging module 108 includes a housing 184 comprising one or more sterilization chambers 186. In one embodiment, the sterilizer 182 includes one or more UV lamps 188 in each of the chambers 186. The UV lamps 188 may be attached to a wall of the housing 184, a door 190 of the housing 184, and/or placed in another location within the chambers 186, as indicated in FIG. 7A. Here, the UV lamps 188 may expose the first couplers 170A to a sterilizing dosage of UV light, such as while the door 190 is closed to contain the UV light within the chambers 186. After the first couplers 170A have been sterilized, the second couplers 170B and their corresponding bags 132 may be connected to the first couplers 170A to form the fluidic couplings 170, and the parenteral solution 120 may be delivered into the bags 132, as indicated in FIG. 7B.

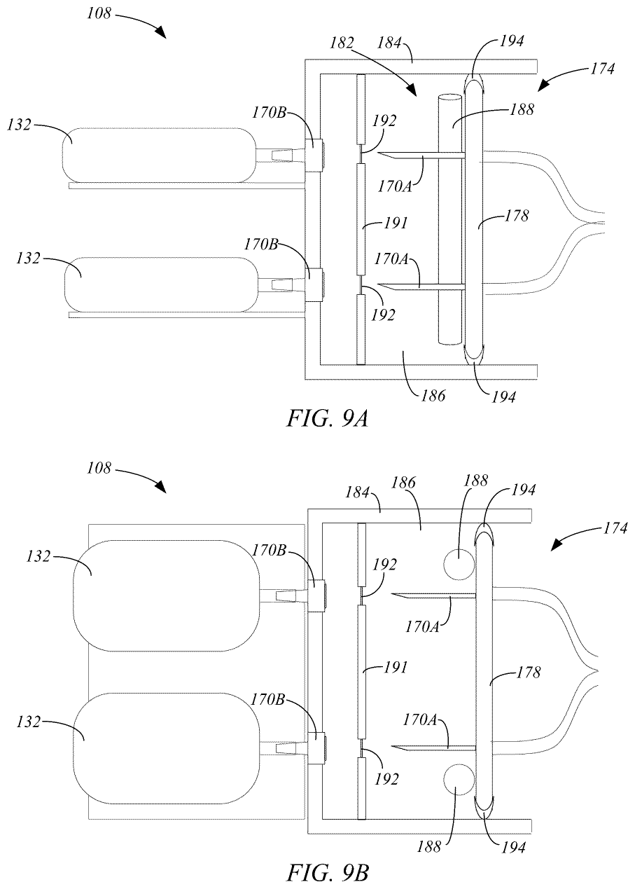

FIGS. 8A and 8B are simplified side views of an example of a bagging module 108 respectively in retracted and filling positions, in accordance with embodiments of the present disclosure. In this example, UV lamps 188 of the sanitizer 182 are contained within a sterilization chamber 186 of a housing 184, such as on the structure 178 supporting the first couplers 170A, on the structure 180 supporting the second couplers 170B, and/or in another location within the chamber 186. The UV lamps 188 expose the first couplers 170A and/or the second couplers 170B to UV light to sanitize the couplers while in the retracted position (FIG. 8A) or during movement of the structure 178 relative to the structure 180 from the retracted position to the filling position (FIG. 8B) by the drive mechanism 174.

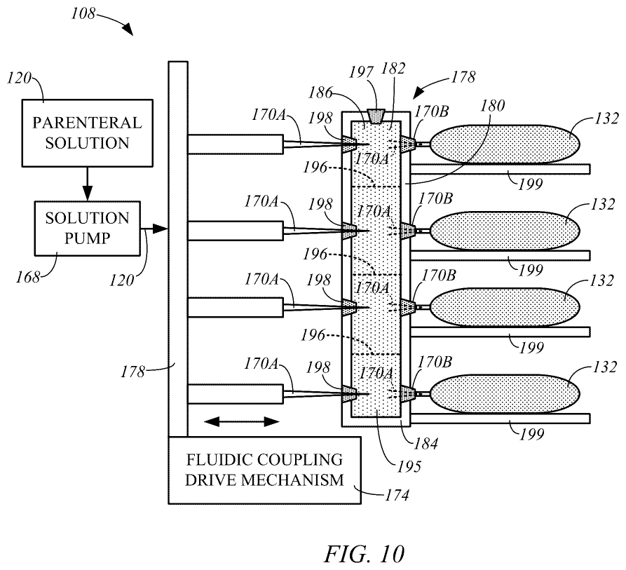

FIGS. 9A and 9B are simplified side and top views of a more detailed example of the bagging module 108 shown in FIGS. 8A and 8B, in accordance with embodiments of the present disclosure. In this example, the first couplers 170A comprise needles and the second couplers 170B comprise ports or septa. One or more UV lamps 188 of the sanitizer 182 supported within the chamber 186 are configured to expose the distal ends of the needles 170A to UV light. In addition, the UV lamps 188 may be configured to expose the septa or ports 170B and other components of the bagging module 108 to a sterilizing dosage of UV light. The UV lamps 188 may be attached to the structure 178 supporting the needles 170A or placed in another suitable location, as discussed above.

In one embodiment, the housing 184 includes a wall 191 containing sealable ports 192, such as septa, each corresponding to one of the needles 170A. The wall 190 may be UV transparent to allow for the sterilization of the ports 192 and the bag septa or ports forming the coupler 170B.

The drive mechanism 174 may be configured to move the structure or wall 178 relative to the housing 184 to transition the needles 170A from their retracted position shown in FIGS. 9A and 9B, to the filling position, in which the needles 170A pierce the ports or septa 192 and 170B and allow the bags 132 to be filled with the solution 120, as represented in FIG. 5. Here, a suitable seal 194 may be provided between the structure 178 and the housing 184 to prevent leakage of UV light and/or fluid.

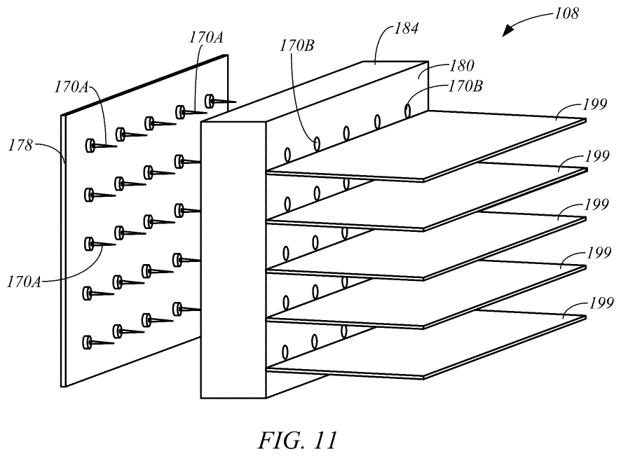

FIG. 10 is a simplified diagram illustrating an example of the bagging module 108 having a sterilizer 182 that utilizes a sterilization fluid 195 to sterilize the first couplers 170A and/or the second couplers 170B. In this example, the first couplers 170A comprise needles and the second couplers 170B comprise suitable sealable ports or septa.

The housing 184 may include one or more sterilization chambers 186 that each contain the sterilization fluid 195. Thus, the housing 184 may contain a single chamber 186 filled with the sterilization fluid 195, or the housing may include walls 196 (phantom lines) that form multiple sterilization chambers 186, each of which may accommodate one or more of the first couplers 170A and/or the second couplers 170B. The one or more chambers 186 may be filled with the sterilization fluid 195 through a port 197. The chambers 186 may be sealed by the sealable ports or septa 170B and housing ports or septa 198.

The drive mechanism 174 may move the structure 178 supporting the needles 170A relative to the housing 184 supporting the ports or septa 170B between the retracted position (needles 170A shown in solid lines) to the filling position (needles 170A shown in phantom lines). During this movement and/or while in the retracted position, the distal ends of the needles 170A extend through the corresponding housing ports or septa 198 and into the sterilization chamber 186, in which they are sterilized by the sterilization fluid 195. The ports or septa 170B may also be sterilized by the sterilization fluid 195.

As the needles 170 are moved further by the drive mechanism 174 toward the ports or septa 170B, the distal ends of the needles 170A pierce the ports or septa 170B and ultimately reach the filling position, in which the parenteral solution 120 may be delivered into the bags 132. After the bags 132 have been filled to a desired volume, they may be disconnected and packaged as discussed above to form the final product 130.

FIG. 11 is an isometric view of an example of the bagging module 108 of FIG. 10, in accordance with embodiments of the present disclosure. In this example, the first couplers 170A (e.g., needles) may be arranged in an array to accommodate the simultaneous or parallel filing of several of the fluid storage bags 132. The housing 184 may include shelves 199 that are arranged to support the bags 132, as indicated in FIGS. 10 and 11.

In some embodiments, the system 100 includes a controller 200 (FIG. 1) that is configured to perform one or more functions described herein. The controller 200 may take on any suitable form. In one embodiment, the controller 200 includes one or more processors 202 that control the components of the system 100 (e.g., components of modules 102, 104, 106 and/or 108) to perform one or more functions or processes described herein (e.g., control the water and solution pumps 117 and 168, the metering devices 122, motor of the needle driving mechanism 174, etc.) in response to the execution of instructions stored in memory 204. The processors 202 of the controller 200 may be components of computer-based systems, and may include control circuits, microprocessor-based engine control systems, and/or programmable hardware components, such as a field programmable gate array (FPGA). The memory 204 represents local and/or remote memory or computer readable media. Such memory comprises any suitable patent subject matter eligible computer readable media that do not include transitory waves or signals. Examples of suitable forms of the memory 204 include hard disks, CD-ROMs, optical storage devices, and/or magnetic storage devices. The controller 200 may include circuitry 206 for use by the one or more processors 202 to receive input signals 208 (e.g., conductivity output from the sensor(s) 123), issue control signals 210 (e.g., control signals to the pumps 117 and 168, the metering devices 122, the needle driving mechanism 174, etc.), and or communicate data 212, such as in response to the execution of the instructions stored in the memory 204.

Although the embodiments of the present disclosure have been described with reference to preferred embodiments, workers skilled in the art will recognize that changes may be made in form and detail without departing from the spirit and scope of the present disclosure.

1. A system for manufacturing parenteral solutions comprising:
a drug dosage module comprising:
a source of one or more parenteral drug solutes; and
one or more metering devices configured to discharge a metered dosage of each of the parenteral drug solutes; and
a mixing module comprising:
a container configured to receive water-for-injection (WFI) and the parenteral drug solutes discharged from the one or more metering devices; and
a mixer configured to mix water-for-injection (WFI) and the parenteral drug solutes in the container to form a parenteral solution.

2. The system according to claim 1, wherein the mixing module comprises at least one conductivity sensor, each configured to sense a conductivity of the parenteral solution in the container and produce a conductivity output that is indicative of the sensed conductivity.

3. The system according to claim 2, further comprising a controller configured to control the discharge of the metered dosage of each of the parenteral drug solutes to the container by the one or more metering devices based on each conductivity output produced by the at least one conductivity sensor.

4. The system according to claim 3, wherein:
the system comprises a water module including a water pump configured to deliver a flow of the WFI to the container; and
the controller is configured to control the delivery of the flow of the WFI to the container by the water pump based on each conductivity output produced by the at least one conductivity sensor.

5. The system according to claim 4, wherein:
the at least one conductivity sensor comprises a plurality of the conductivity sensors, each positioned at a different height within the container; and
the controller controls the discharge of the metered dosage of each of the parenteral drug solutes by the one or more metering devices and the flow of the WFI to the container by the water pump based on the conductivity outputs.

6. The system according to claim 5, wherein the water module comprises a water purifier configured to filter or sterilize a flow of water to produce the flow of the WFI.

7. The system according to claim 6, wherein the water purifier comprises a water filter, through which the flow of water travels, and/or an ultraviolet (UV) sterilization device comprising one or more UV light sources configured to expose the flow of water to UV light.

8. The system according to claim 1, further comprising a bagging module configured to deliver a flow of the parenteral solution into each of one or more fluid storage bags through a corresponding fluidic coupling.

9. The system according to claim 8, wherein each fluidic coupling comprises a first coupler configured to receive the flow of the parenteral solution and a second coupler attached to the fluid storage bag, the first and second couplers configured to cooperate to form the fluidic coupling.

10. The system according to claim 9, wherein:
the first coupler comprises a needle, and the second coupler comprises a septum;
the first and second couplers comprise cooperating tubing fittings; or
the first and second couplers comprise cooperating Luer fittings.

11. The system according to claim 10, wherein the bagging module comprises a solution pump configured to drive the flow of the parenteral solution from the container into each of the one or more fluid storage bags through the corresponding fluidic coupling.

12. The system according to claim 11, wherein the bagging module comprises a fluidic coupling drive mechanism configured to move each of the first couplers relative to the second couplers between a retracted position, in which each of the first couplers is recessed from the corresponding second coupler, and a filling position, in which each of the first couplers cooperates with the corresponding second coupler to form the fluidic couplings.

13. The system according to claim 9, wherein the bagging module further comprises a sterilizer configured to sterilize the first coupler and/or the second coupler of each fluidic coupling comprising:
a first sterilizer comprising:
at least one sterilization chamber, wherein the first coupler and/or the second coupler of each fluidic coupling is contained in one of the sterilization chambers; and
one or more UV lamps contained in each of the sterilization chambers and configured to expose the first coupler and/or the second coupler of each fluidic coupling to UV light; or
a second sterilizer comprising:
at least one sterilization chamber, each containing a sterilization fluid; and
the first coupler and/or the second coupler of each fluidic coupling is contained in one of the sterilization chambers and the sterilization fluid.

14. (canceled)

15. (canceled)

16. The system according claim 12, wherein:
the bagging module comprises a plurality of the fluid storage bags and a plurality of the fluidic couplings;
the first coupler of each fluidic coupling comprises a needle;
the second coupler of each fluidic coupling comprises a bag septum fluidically coupled to an interior cavity of the corresponding fluid storage bag; and
the fluidic coupling drive mechanism is configured to move the plurality of needles relative to the corresponding bag septa between a retracted position, in which each of the needles is recessed from the corresponding bag septum, and a filling position, in which each of the needles pierces the corresponding bag septum and is configured to deliver the flow of the parenteral solution to the interior cavity of the corresponding fluid storage bag.

17. (canceled)

18. A bagging module configured to fill one or more fluid storage bags with parenteral solution, the bagging module comprising:
a source of parenteral solution;
one or more fluid storage bags, each having an interior cavity; and
a fluidic coupling corresponding to each fluid storage bag to facilitate delivery of a flow of the parenteral solution from the source into the one or more fluid storage bags.

19. The bagging module according to claim 18, wherein each fluidic coupling comprises a first coupler configured to receive the flow of the parenteral solution and a second coupler attached to the fluid storage bag, the first and second couplers configured to cooperate to form the fluidic coupling.

20. The bagging module according to claim 19, wherein:
the first coupler comprises a needle, and the second coupler comprises a septum;
the first and second couplers comprise cooperating tubing fittings; or
the first and second couplers comprising cooperating Luer fittings.

21. The bagging module according to claim 20, wherein the bagging module comprises a solution pump configured to drive the flow of the parenteral solution from the source into each of the one or more fluid storage bags through the corresponding fluidic coupling.

22. The bagging module according to claim 21, wherein the bagging module comprises a fluidic coupling drive mechanism configured to move each of the first couplers relative to the second couplers between a retracted position, in which each of the first couplers is recessed from the corresponding second coupler, and a filling position, in which each of the first couplers cooperates with the corresponding second coupler to form the fluidic couplings.

23. The bagging module according to claim 18, wherein the bagging module further comprises a sterilizer configured to sterilize the first coupler and/or the second coupler comprising:
a first sterilizer comprising:
at least one sterilization chamber, wherein the first coupler and/or the second coupler of each fluidic coupling is contained in one of the sterilization chambers; and
one or more UV lamps contained in each of the sterilization chambers and configured to expose the first coupler and/or the second coupler of each fluidic coupling to UV light; or
a second sterilizer comprising:
at least one sterilization chamber, each containing a sterilization fluid; and
the first coupler and/or the second coupler of each fluidic coupling is contained in one of the sterilization chambers and the sterilization fluid.

24. (canceled)

25. (canceled)
